(19) United States
(12) Patent Application Publication
(10) Pub. No.: US 2015/0005252 A1
(43) Pub. Date: Jan. 1, 2015

(54) COMBINATION THERAPY FOR THE TREATMENT OF CANCER

(71) Applicant: TILTAN PHARMA LTD., Jerusalem (IL)

(72) Inventors: Dafna Feitelberg, Kiryat Ono (IL); Tzivia Berkman, Tel-Aviv (IL); Shmuel Ben-Sasson, Jerusalem (IL); Dan Goldstaub, Even Yehuda (IL)

(21) Appl. No.: 14/371,222

(22) PCT Filed: Jan. 8, 2013
(86) PCT No.: PCT/IL2013/050021

§ 371 Date: Jul. 9, 2014

Related U.S. Application Data
(60) Provisional application No. 61/584,385, filed on Jan. 9, 2012.

(51) Int. Cl. A61K 31/7068; A61K 31/655; A61K 31/4164; A61K 31/196; A61K 31/675

(52) U.S. Cl. CPC A61K 31/7068; A61K 31/196; A61K 31/675; A61K 31/4164; A61K 31/655
USPC 514/49

(57) ABSTRACT

The present invention provides a combination comprising: a composition comprising at least one non-steroidal anti-inflammatory agent and at least one cytotoxic agent; and a composition comprising at least one ribonucleotide reductase inhibitor. The invention further includes kits comprising a combination of the invention and methods and uses of a combination of the invention for the treatment of cancer treatment of cancer, including solid tumors or tumor metastasis.

### FIELD OF THE INVENTION

This invention relates to the field of combination anti-cancer therapy and/or prevention.

### BACKGROUND OF THE INVENTION

Cancer generally refers to one of a group of diseases caused by the uncontrolled, abnormal growth of cells that can spread to adjoining tissues or other parts of the body. Cancer cells can form a solid tumor, in which the cancer cells are massed together, or exist, as dispersed cells, as in leukemia. Normal cells divide until maturation is attained and then only as necessary for replacement of damaged or dead cells. Cancer cells are often referred to as “malignant”, because they divide endlessly, eventually crowding out nearby tissues and spreading to other parts of the body. The tendency of cancer cells to invade and spread from one organ to another or from one part of the body to another distinguishes them from benign tumor cells, which overgrow but do not spread to other organs or parts of the body. Malignant cancer cells eventually metastasize and spread to other parts of the body via the bloodstream or lymphatic system, where they can multiply and form new tumors. This sort of tumor progression makes cancer a deadly disease.

Although there have been great improvements in the diagnosis and treatment of cancer, many people die from cancer each year, and their deaths are typically due to metastases and cancers that are resistant to conventional therapies.

Most drug-mediated cancer therapies rely on poisons, called cytotoxic agents, selective for dividing cells. These drugs are effective because cancer cells generally divide more frequently than normal cells. However, such drugs almost inevitably do not kill all of the cancer cells in the patient. One reason is that cancer cells can acquire mutations that confer drug resistance. Another is that not all cancer cells divide more frequently than normal cells, and slowly-dividing cancer cells can be as, or even more, insensitive to such cytotoxic agents as normal cells. Some cancer cells divide slowly, because they reside in a poorly vascularized, solid tumor and are unable to meet the needs required for cell division. For example, cytotoxic agents such as cyclophosphamide have been used to treat cancer.

Although cancer chemotherapy has advanced dramatically in recent years, treating cancers with a single agent has had limited success. Firstly, any single agent may only target a subset of the total population of malignant cells present, leaving a subpopulation of cancerous cells to continue growing. Secondly, cells develop resistance upon prolonged exposure to a drug. Combination therapies, which employ two or more agents with differing mechanisms of action and differing toxicities, have been useful for circumventing drug resistance and increasing the target cell population, but have not proven effective in the treatment of all cancers. In addition, certain combinations of agents may be synergistic: their combined effect is larger than that predicted based on their individual activities. Thus, combining different agents can be a powerful strategy for treating cancer.

One problem with cytotoxic agents which function by disrupting cell division is that they do not discriminate between normal and malignant cells: any dividing cell is a potential target for their action. Thus, cell populations which normally exhibit high levels of proliferation (such as bone marrow) are affected, leading to the toxic side effects commonly associated with cancer treatments.

With only a few exceptions, no single drug or drug combination is curative for most cancers. Thus, there is a continuous need for new combinations therapies that can delay the growth of life-threatening tumors and/or improve quality of life by further reducing tumor load, providing high survival rate and relatively low toxicity.

### SUMMARY OF THE INVENTION

In the first aspect the present invention provides a combination comprising:
a. a composition (also referred to as “composition (a)”), comprising at least one non-steroidal anti-inflammatory agent and at least one cytotoxic agent; and
b. a composition (also referred to as “composition (b)”), comprising at least one ribonucleotide reductase inhibitor.

It was surprisingly found by the inventors of the present application that administration of a combination of the invention can effectively and satisfactorily treat cancer in a subject, to the extent of either lowering the number of, reducing the size or number of, slow the progression of, preventing the development of or eradicating of malignant tumors in said subject with relatively low toxicity parameters and effects and achieving high survival rate of said subject.

The combination of the invention should be understood to encompass any type of combination of composition (a) and composition (b) as defined herein above and below. Thus, in some embodiments, composition (a) and composition (b) are two separate compositions (dosage forms), forming a combination of the invention when administered either simultaneously or concomitantly. In other embodiments, composition (a) and composition (b) form a single combined dosage form.

Composition (a) comprises at least one non-steroidal anti-inflammatory agent and at least one cytotoxic agent.

The term “non-steroidal anti-inflammatory agent (drug)” should be understood to encompass any agent, not including a steroid agent, capable of reducing and/or inhibiting and/or preventing an inflammatory disease or disorder cased by either response to infection, injury, irritation, or surgery.

In some embodiments said at least one non-steroidal anti-inflammatory agent is a COX-1 or COX-2 inhibitor. In other embodiments said non-steroidal anti-inflammatory agent is selected from a COX-1 inhibitor, a COX-2 inhibitor and a non-selective COX-1 and COX-2 inhibitor.

In further embodiments said at least one non-steroidal anti-inflammatory agent is selected from the group consisting of diclofenac, piroxicam, indomethacin and any combination thereof.

The term “cytotoxic agent” should be understood to encompass any agent (from a natural, synthetic or semi-synthetic source) that has a degree of cell toxicity and us used in the treatment of abnormal and uncontrolled progressive cellular growth. A cytotoxic agent acts as an angiogenesis inhibitor when administered at a low dose.

Non limiting examples of cytotoxic agents include the alkylating agents cyclophosphamide (CTX) (Bristol-Meyers Squibb), ifosfamide (Bristol-Meyers Squibb), chlorambucil (Glaxo Wellcome), and carmustine (Bristol-Meyers Squibb); the anti-metabolites cytarabine (Pharmacia & Upjohn), 6-mercaptopurine (Glaxo Wellcome), 6-thioguanine (Glaxo Wellcome), and methotrexate (Immunex); the antibiotics doxorubicin (Pharmacia & Upjohn), daunorubicin (NeXstar), and mitoxantrone (Immunex); and miscellaneous agents such as vincristine (Lilly), vinblastine (Lilly), and paclitaxel (Bristol-Meyers Squibb), including any metabolites and prodrugs thereof and any combinations thereof.

In some embodiments said at least one cytotoxic agent is selected from the group consisting of: cyclophosphamide, ifosfamide, cytarabine, 6-mercaptopurine, 6-thioguanine, vincristine, doxorubicin, daunorubicin, chlorambucil, carmustine, vinblastine, methotrexate, mitoxantrone, paclitaxel or their pharmaceutically acceptable salts and any combination thereof including any metabolites and prodrugs thereof and any combinations thereof.

In further embodiments, said at least one cytotoxic agent is cyclophosphamide or ifosfamide.

The term “ribonucleotide reductase inhibitor” should be understood to encompass any agent capable of inhibiting (to any extent, i.e. qualitatively or quantitatively) the ribonucleotide reductase enzyme catalyzing the formation of deoxyribonucleotides from ribonucleotides.

In some embodiments, said at least one ribonucleotide reductase inhibitor is selected from the group consisting of fludarabine, cladribine, gemcitabine, tezacitabine, triapine, motexafin gadolinium, hydroxyurea, gallium maltolate, gallium nitrate and any combination thereof. Said ribonucleotide reductase inhibitor include also any metabolites and prodrugs thereof.

In further embodiments, said at least one ribonucleotide reductase inhibitor is gemcitabine (4-amino-1-(2-deoxy-2,2-difluoro-β-D-erythro-pentofuranosyl)pyrimidin-2(1H)-on), or any pharmaceutically acceptable salts thereof. In other embodiments, said at least one ribonucleotide reductase inhibitor is a prodrug of gemcitabine (see Zhao C. et al. Chem. Biol. Drug Des. 2012, 80:479-488)), such as for example LY2334737 (Eli Lilly).

In some embodiments composition (a) of the invention further comprises levamisol ((S)-6-Phenyl-2,3,5,6-tetrahydroimidazo[2,1-b][1,3]thiazole), or any pharmaceutically acceptable salts thereof.

In some embodiments composition (a) of the invention further comprises at least one NFkB inhibitor. The term “NFkB inhibitor” as used herein relates to any agent used for the inhibition of the Nuclear Factor kappa B (NFkB) intracellular transcription factor. In some embodiments, the NFkB inhibitor is selected from sulfasalazine, rapamycin, caffeic acid phenethylester, SN50 (a cell-permeable inhibitory peptide), parthenolide, triptolide, wedelolactone, lactacystin, MG-132 [Z-Leu-Leu-Leu-H] and any combination thereof. In some other embodiments, said NFkB inhibitor is sulfasalazine or rapamycin.

In some embodiments composition (a) of the invention further comprises at least one H2-blocker. In some embodiments said H2-blocker is selected from the group consisting of cimetidine, ranitidine, famotidine, nizatidine and any combination thereof.

In some embodiments composition (a) of the invention further comprises at least one agent that enhances intracellular accumulation of NADH+H⁺. In some embodiments said agent enhancing intracellular accumulation of NADH+H⁺ is a poly-alcohol. In further embodiments, said poly-alcohol is selected from the group consisting of xylitol, mannitol, sorbitol, arabinol, iditol and any combination thereof. In yet further embodiments, said poly-alcohol is xylitol.

In some embodiments composition (a) of the invention further comprises at least one inhibitor of a matrix metalloproteinase As used herein, the phrase “matrix metalloproteinase (MMP) inhibitor” relates to any chemical compound that inhibits by at least 5%, the hydrolytic activity of at least one matrix metalloproteinase enzyme that is naturally occurring in a mammal. The MMP inhibitor may be any MMP inhibitor known in the art, such as for example AG-3340, RO 32-3555, RS 13-0830, Tissue Inhibitors of metalloproteinases (TIMPs) (e.g. TIMP-1, TIMP-2, TIMP-3, or TIMP-4), alpha 2-macroglobulin, tetracyclines (e.g., tetracycline, minocycline, and doxycycline), hydroxamates (e.g. batimastat, marimistat and trocade), chelators (e.g., EDTA, cysteine, acetylcysteine, D-penicillamine, and gold salts), synthetic MMP fragments, succinyl mercaptopurines, phosphonamidates, and hydroxaminic acids.

In some embodiments composition (a) of the invention further comprises at least one inhibitor of a pro-angiogenic factor. The term “inhibitor of pro-angiogenic growth factor” relates to any agent that is used to inhibit the signaling of known pro-angiogenic factors such as VEGF, FGF or PDGF. Without wishing to be bound by theory, it was shown that these agents can act extracellularly, by the inhibition of the interaction of an angiogenic factor with its receptor or can act intracellularly via the inhibition of the protein-kinase activity of the corresponding receptors. Non limiting examples of these agents include anti-VEGF or anti-VEGF-Receptor antibodies or inhibitors of the protein-kinase domain of VEGF-R, FGF-R or PDGF-R.

In some embodiments composition (a) of the invention further comprises at least one redox quinone. In some embodiments said redox quinone is Vitamin K₃. In further embodiments said Vitamin K₃ is selected from a group consisting of menadione, menadione sodiumbisulfite, and any combination thereof. Quinones are compounds having a fully conjugated cyclic dione structure, such as that of benzoquinones, derived from aromatic compounds by conversion of an even number of —CH═ groups into —C(═O)— groups with any necessary rearrangement of double bonds (polycyclic and heterocyclic analogues are included). Quinones are known for their ability to induce oxidative stress through redox cycling, hereby referred to as “Redox quinones” (Powis G., Free Radic. Biol. Med. 6:63-101 (1989)). Pharmaceutically acceptable redox quinones such as Vitamin K₃ have special therapeutic value since they are required for the bioactivation of proteins involved in hemostasis. Vitamin K₃ is a redox quinone, known as a prothrombogenic agent, mainly in supplement of veterinary diet. Studies have shown that Vitamin K₃ has failed to demonstrate beneficial anti-cancer properties (Tetef M. et al. J. Cancer Res. Clin. Oncol. 121:103-6 (1995)).

In some embodiments composition (a) of the invention further comprises at least one retinoid. In some embodiments, said retinoid is all-trans-retinoic-acid (ATRA). The term “retinoid” as used herein relates to a class of chemical compounds that are related chemically to vitamin A. As used herein, a retinoid component of the invention is any compound which acts through and/or binds to retinoic acid receptors (RARs) or to retinoid X receptors (RXRs).

In some embodiments of the invention said composition (a) further comprises at least one of the following: levamisol, an NFkB inhibitor, an H2-blocker, at least one agent that enhances intracellular accumulation of NADH+H⁺, an inhibitor of a matrix metalloproteinase, an inhibitor of a pro-angiogenic factor, a redox quinone, a retinoid, including any combination thereof, any pharmaceutically acceptable salts thereof.

In some embodiments, components of composition (a) are administered in a single dosage from. In other embodiments, components of composition (a) are administered separately either simultaneously or concomitantly with each other and with composition (b).

A composition of the invention may further include at least one pharmaceutically actable carrier. In the context of the present invention the term “pharmaceutically acceptable carrier” relates to pharmaceutically-acceptable, nontoxic carriers or diluents, which are defined as vehicles commonly used to formulate pharmaceutical compositions for animal or human administration. Such carriers may include, however not limited to, buffering agents, solubilizing agents, stabilizing agents or taste additives.

A composition of the invention may further include at least one pharmaceutically acceptable auxiliaries, and optionally other therapeutic agents. The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrants, lubricants, colorants, flavouring agents, anti-oxidants, and wetting agents.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

The invention further includes a pharmaceutical composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators.

The exact dose and regimen of administration of the composition will necessarily be dependent upon the therapeutic or nutritional effect to be achieved and may vary with the particular formula, the route of administration, and the age and condition of the individual subject to whom the composition is to be administered.

The present invention further provides a formulation consisting of an aqueous or oily suspension or solution comprising a pharmaceutical composition of the invention. In a further embodiment of the present invention, the formulation further comprises a flavoring agent (e.g. menthol, anethol and/or salt). In another embodiment of the present invention, part of the constituents of the aqueous or oily suspension or solution of the formulation may be supplied in a dry form and reconstituted (e.g. solubilized) prior to oral administration.

In some embodiments, the formulation is formulated for oral administration. Such oral administration may allow for treatment to take place, for example, at the patient's home.

In some embodiments of the present invention compositions may be provided as sustained release or timed release formulations. The carrier or diluent may include any sustained release material known in the art, such as glyceryl monostrearate or glyceryl distearate, alone or mixed with a wax. Micro-encapsulation may also be used. The timed release formulation can provide a pharmaceutical composition of immediate and pulsed release throughout the day. The diluent is selected so as not to affect the biological activity of a pharmaceutical composition of the invention. Examples of such diluents are distilled water, physiological saline, Ringer's solution, dextrose solution, and Hank's solution.

A pharmaceutical composition or formulation of the subject invention may include carriers, adjuvants and emulsifiers such as poloxamers, or nontoxic, non-therapeutic, non-immunogenic stabilizers and the like. Effective amounts of such diluent or carrier will be those amounts which are effective to obtain a pharmaceutically acceptable formulation in terms of solubility of components, biological activity, and the like.

In some embodiments, the formulations include a controlled-release device or composition where one or several of the components comprised in a pharmaceutical composition of the invention are being released in a delayed fashion. Such formulation may be in the form of a tablet (or a pill) which releases different doses of components comprised in a pharmaceutical composition of the invention, in different time intervals after being administered orally.

A pharmaceutical composition of the invention may be formulated in a solid, semi-solid, or liquid form such as, e.g. suspensions, aerosols, or the like or any other formulation known to a person skilled in the art. In some embodiments, the compositions are administered in unit dosage forms suitable for single administration of precise dosage amounts. The compositions may also include, depending on the formulation desired, pharmaceutically-acceptable carriers as defined above.

A pharmaceutical composition of the present invention or each component thereof can thus be administered by any means known in the art, such as oral (including buccal and sublingual), rectal, vaginal, nasal, topical, transdermal, or parenteral (including subcutaneous, intramuscular, intravenous, intrasynovial, intraperitoneal and intradermal) administration.

Pharmaceutical compositions, methods and systems of the present invention may be used either alone, or in conjunction with other cancer treatment methods known to those of skill in the art. Such methods may include, but are not limited to chemotherapy, radiation therapy or surgery. The administration of a pharmaceutical composition of the present invention may be conducted before, during or after other cancer therapies. In addition, a pharmaceutical composition of the present invention may be administered concurrently with other cancer treatments known to those of skill in the art.

Typically, oral administration requires a higher dose than intravenous administration. Thus, the administration route will depend upon the situation: the skilled artisan must determine which form of administration is best in a particular case, balancing dose needed versus the number of times per month administration is necessary.

In some embodiments of the present invention, a pharmaceutical composition of the invention may be administered in a single dosage form comprising all the components together. In another embodiment, at least one component of a pharmaceutical composition of the invention may be separately administered, simultaneously or sequentially.

In a further aspect the invention provides a combination as defined herein above, for use in the treatment of cancer.

In yet another aspect the invention provides a method of treating cancer in a mammal comprising administering to said mammal a combination according to the invention, as defined herein above.

The term “cancer” as referred to in the present invention relates to any neoplastic disease which is characterized by abnormal and uncontrolled cell division causing malignant growth or tumor. Cancer cells, unlike benign tumor cells, exhibit the properties of invasion and metastasis and are highly anaplastic. Cancer includes the two broad categories of carcinoma and sarcoma. Non-limiting examples of types of cancer disease include lung cancer (e.g. adenocarcinoma and including non-small cell lung cancer), pancreatic cancers (e.g. pancreatic carcinoma such as, for example exocrine pancreatic carcinoma), colon cancers (e.g. colorectal carcinomas, such as, for example, colon adenocarcinoma and colon adenoma), prostate cancer including the advanced disease, hematopoietic tumors of lymphoid lineage (e.g. acute lymphocytic leukemia, B-cell lymphoma, Burkitt's lymphoma), myeloid leukemias (for example, acute myelogenous leukemia (AML)), thyroid follicular cancer, myelodysplastic syndrome (MDS), tumors of mesenchymal origin (e.g. fibrosarcomas and rhabdomyosarcomas), melanomas, teratocarcinomas, neuroblastomas, gliomas, glioblastoma, benign tumor of the skin (e.g. keratoacanthomas), breast carcinoma (e.g. advanced breast cancer), kidney carcinoma, ovary carcinoma, bladder carcinoma and epidermal carcinoma.

In some embodiments, said cancer is a solid tumor (i.e. essentially solid neoplasmic growth, with low liquid content that is other than a cyst) or tumor metastasis (i.e. at its metastatic stage of disease).

In further embodiments, said cancer is selected from lung cancer, pancreatic cancer, colon cancer, prostate cancer, hematopoietic tumors of lymphoid lineage, myeloid leukemias, thyroid follicular cancer, myelodysplastic syndrome, tumors of mesenchymal origin, melanoma, teratocarcinoma, neuroblastoma, glioma, glioblastoma, benign tumor of the skin, breast carcinoma, kidney carcinoma, ovary carcinoma, bladder carcinoma, epidermal carcinoma and any combination thereof.

The term “treatment of cancer” as used in the context of the present invention relates to any kind of change in the disease state or condition of a subject in need thereof including any degree of: a decrease in tumor size; decrease in rate of tumor growth; stasis of tumor size; decrease in the number of metastasis; decrease in the number of additional metastasis; decrease in invasiveness of the cancer; decrease in the rate of progression of the tumor from one stage to the next, inhibition of tumor growth in a tissue of a mammal having a malignant cancer, control of establishment of metastases, inhibition of tumor metastases formation, regression of established tumors as well as decrease in the angiogenesis induced by the cancer. The term “treatment of cancer” can also refer to prophylactic treatment, such for example the prevention of cancer reoccurs after previous treatment (including surgical removal) and prevention of cancer in an individual prone (genetically, due to life style, chronic inflammation and so forth) to develop cancer.

The term “administering” or its other lingual forms as used in the context of the present invention relates to the path by which a pharmaceutically active component, a drug, fluid or other substance is brought into contact with the body of a subject. The pharmaceutical composition is transported from the site of entry to the part of the body where its action is desired to take place. According to one embodiment of the present invention, said administering may be achieved via any medically acceptable means suitable for a pharmaceutical composition of the invention or any component thereof, including oral, rectal, vaginal, nasal, topical, transdermal, or parenteral (including subcutaneous, intramuscular, intrasynovial, intraperitoneal, intradermal and intravenous) administration.

In therapeutic applications, the dosages and administration schedule of components of a pharmaceutical composition of the invention may vary depending on the component, the age, weight, and clinical condition of the recipient patient, and the experience and judgment of the clinician or practitioner administering the therapy, among other factors affecting the selected dosage. Generally, the dose and administration scheduled should be sufficient to result in slowing and/or regressing, the growth of the tumor(s) and may also cause complete regression of the cancer. In some cases, regression may be monitored via direct imaging (e.g. MRI) or by a decrease in blood levels of tumor specific markers. An effective amount of the pharmaceutical composition is that which provides a medical benefit as noted by the clinician or other qualified observer. Regression of a tumor in a patient is typically measured with reference to the diameter of a tumor. Decrease in the diameter of a tumor indicates regression. Complete regression is also indicated by failure of tumors to reoccur after treatment has stopped. The present invention allows for the administration of a pharmaceutical composition of the present invention, either prophylactically or therapeutically or in the context of adjuvant or neo-adjuvant treatment.

When provided prophylactically, a pharmaceutical composition of the invention may be administered in advance of any symptom. Prophylactic administration of pharmaceutical compositions may serve to prevent or inhibit cancer. A pharmaceutical composition of the invention may prophylactically be administered to a patient with, for example, a family history of cancer. The risk for developing cancer may be determined by measuring levels of cancer marker proteins in the biological fluids (i.e. blood, urine) of a patient or by genetic markers. Alternatively, administration of a pharmaceutical composition of the invention may be administered to a patient with rising cancer marker protein levels. Such markers include, for example, rising PSA, CEA, thymosin β-15, thymosin β-16, calcitonin, and matrix metalloproteinase (MMP). When provided prophylactically, the dose of a pharmaceutical composition of the invention may be reduced to the appropriate prophylactic dosage.

When provided therapeutically, a pharmaceutical composition of the invention may be administered at (or after) the onset of a symptom or indication of a cancer. Thus, a pharmaceutical composition of the present invention may be provided either prior to the anticipated tumor growth at a site or after the malignant growth has begun at a site.

In all aspects and embodiments of the invention when referring to a subject in need of a treatment of the invention, it should be understood to relate to a “mammal”, a warm blooded vertebrate animals characterized by the presence of mammary glands, which produce milk in females for the nourishment of young, and in addition are covered with hair or fur. In some embodiments, said mammal may be selected from the group consisting of a human, a cat, a dog and a horse.

In further embodiments of the invention said combination of the invention may be administered to a subject in any predetermined dosing regimen over a predetermined period of time, wherein composition (a) comprising at least one non-steroidal anti-inflammatory agent and at least one cytotoxic agent and composition (b) comprising a ribonucleotide reductase inhibitor; are administered to said subject in any administration sequence during said predetermined period of time.

In some embodiments a combination of the invention is administered in a regiment wherein composition (a) is administered to said subject before (prior to) administration of composition (b) (either on the same day of administration of composition (a) or on a separate day of treatment).

In some embodiments a combination of the invention is administered in a regiment wherein composition (a) is administered on the first day of a predetermined treatment cycle and composition (b) is administered on the second day of treatment.

In some embodiments a combination of the invention is administered in a regiment wherein composition (b) is administered to said subject before (prior to) administration of composition (a) (either on the same day of administration of composition (b) or on a separate day of treatment).

In further embodiments a combination of the invention is administered in a regiment wherein composition (b) is administered on the first day of a predetermined treatment cycle and composition (b) is administered on the second day of treatment.

In some embodiments, dosing regimen of a combination of the invention comprises administration of composition (a) twice a week and administration of composition (b) is twice a week.

In further embodiments, dosing regimen of a combination of the invention comprises administration of composition (a) twice a week and administration of composition (b) once a week.

In other embodiments, a combination of the invention further comprises composition (c) comprising an H2-blocker and an NFkB inhibitor.

In further embodiments, dosing regimen of a combination of the invention comprises administration of composition (a), composition (b) and composition (c) on separate days of a weekly treatment cycle.

In another one of its aspects the invention provides a combination comprising:
a. a composition (also referred to as “composition (a)”), comprising at least one non-steroidal anti-inflammatory agent and at least one cytotoxic agent;
   In some embodiments composition (a) further comprises at least one of the following: levamisol, an NFkB inhibitor, an H2-blocker, at least one agent that enhances intracellular accumulation of NADH→H⁺, an inhibitor of a matrix metalloproteinase, an inhibitor of a pro-angiogenic factor, a redox quinone, a retinoid, including any combination thereof, any pharmaceutically acceptable salts thereof or any prodrugs thereof;
b. a composition (also referred to as “composition (b)”), comprising at least one ribonucleotide reductase inhibitor; and optionally
c. a composition (also referred to as “composition (c)”) comprising an H2-blocker and an NFkB inhibitor.

In another one of its aspects the invention provides a combination comprising:
a. a composition (also referred to as “composition (a)”), comprising at least one non-steroidal anti-inflammatory agent and at least one cytotoxic agent;
   In some embodiments composition (a) further comprises at least one of the following: levamisol, an NFkB inhibitor, an H2-blocker, at least one agent that enhances intracellular accumulation of NADH+H⁺, an inhibitor of a matrix metalloproteinase, an inhibitor of a pro-angiogenic factor, a redox quinone, a retinoid, including any combination thereof, any pharmaceutically acceptable salts thereof or any prodrugs thereof;
b. a composition (also referred to as “composition (b)”), consisting of at least one ribonucleotide reductase inhibitor; and optionally
c. a composition (also referred to as “composition (c)”) consisting of an H2-blocker and an NFkB inhibitor.

In further embodiments, combination of the invention is administered in the following dosing regimen:

| Day | Composition administered |
|---|---|
| 1 | Composition (b) |
| 2 | Composition (a) |
| 3 | Composition (c) |
| 4 | Composition (c) |
| 5 | Composition (a) |
| 6 | Composition (c) |
| 7 | Composition (c) |

In a further aspect the invention provides a combination kit comprising: a first container comprising composition (a) as defined hereinabove; a second container comprising composition (b) as defined hereinabove; and instructions for administration.

The term “container” as used herein refers to any receptacle capable of holding at least one component of a composition of the invention. Such a container may be any jar, vial or box known to a person skilled in the art and may be made of any material suitable for the components contained therein and additionally suitable for short or long term storage under any kind of temperature.

In some embodiments said kit of the invention further comprised a third container comprising composition (c) comprising an H2-blocker and an NFkB inhibitor.

In further embodiments, said instructions of a kit of the invention provide instructions for administration of said combination in the following weekly cycle:

| Day | Composition administered |
|---|---|
| 1 | Composition (b) |
| 2 | Composition (a) |
| 3 | Composition (c) |
| 4 | Composition (c) |
| 5 | Composition (a) |
| 6 | Composition (c) |
| 7 | Composition (c) |

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows the percentage of tumor bearing animals of each group treated according to schedule in Example 1.

FIG. 2 shows the average tumor weight of tumor-bearing animals of each group treated according to schedule in Example 1.

FIG. 3 shows animal survival of each group at each day during treatment schedule according to Example 1.

FIG. 4 shows animal body weight change at each day during treatment schedule according to Example 1.

FIGS. 5A-5E illustrates representative tumors found at the end of each treatment schedule for each treatment group.

### DETAILED DESCRIPTION OF EMBODIMENTS

### EXAMPLE 1

Duration of the Experimental Period

Total study duration was 18 days (Treatment period: 14 days, i.e. two weekly treatment schedules). Tumour cells inoculation day was registered as “Day 1”. The compositions were administered IP six days per week starting on Day 4.

Tumor Cells: Mouse pancreatic-adenocarcinoma cell-line PANC02 (a kind gift from Dr A. Marten, University of Heidelberg, Germany).

Test Animals

CB6F1 female mice, 21.1 g average body weight, 10 mice per treatment group.

Treatment Groups and Compositions

Five test groups were conducted, each administered with a different treatment composition and schedule as detailed in Tables 1 and 2 below.

Treatment Groups were administered with either vehicle alone (Group 1), a combination of TL-118 and Gemcitabine (Groups 2 and 3), Gemcitabine alone 9 Group 4) and TL-118 alone (Group 5).

TL-118 refers to a combination treatment as detailed in Table 2 below.

TABLE 1
Treatment Groups and Compositions

| Group | Composition | Contents |
|---|---|---|
| Group 1 | Vehicle | 25% Hypdroxypropyl β Cyclodextrin + 7% |
| (Vehicle of TL-118) | | PEG400 + 1% Na-Saccharin + 1% Strawberry |
| | | flavor + 1% Ethanol, in aqueous medium |
| Group 2 | Composition (a) | TL-118 (see Table 2) |
| (TL-118 + GEM) | Composition (b) | Gemcitabine 60 mg/kg, twice a week |
| Group 3 | Composition (b) | Gemcitabine 60 mg/kg, twice a week |
| GEM + TL-118 | Composition (a) | TL-118 (see Table 2) |
| Group 4 | Composition (b) | Gemcitabine 60 mg/kg, twice a week |
| (GEM) | | |
| Group 5 | Composition (a) | TL-118 (see Table 2) |
| (TL-118) | | |

TABLE 2
TL-118 Components and Dosing Schedule

| | Treatment Day | | | | | | |
|---|---|---|---|---|---|---|---|
| TL-118 components | 1^{st} | 2^{nd} | 3^{rd} | 4^{th} | 5^{th} | 6^{th} | Sat. |
| Cimetidine 60 mg/kg | ✓ | ✓ | ✓ | ✓ | ✓ | ✓ | |
| Cyclophosphamide 60 mg/kg | ✓ | | | ✓ | | | |
| Diclofenac 30 mg/kg | ✓ | | | ✓ | | | |
| Sulfasalazine 50 mg/kg | ✓ | | | ✓ | | | |
| Sulfasalazine 150 mg/kg | | ✓ | ✓ | | ✓ | ✓ | |

In Groups 2 and 5 the 1^{st} day of TL-118 was Wed. while for Group 3 1^{st} day of TL-118 was Thur. No treatment was given on Sat (see also Table 3 below).

In Group 2 the treatment started with TL-118 and Gemcitabine was administered on the following day while in Group 3 the treatment started with Gemcitabine administration then TL-118 initiation on the following day (see Table 3 below).

Preparation of Cells for Injection

The cells were removed from flasks using trypsin. 60 million (60×10⁶) cells were counted and re-suspended in 4.5 ml phosphate buffered saline. Before cell spin-down, cells were strained through cell strainer according to the following procedure:
1. Strainer was placed on 50 ml tube;
2. Strainer was washed with 5 ml phosphate buffered saline;
3. Cells detached from flasks in RPMI growth medium were poured over strainer;
4. Strainer was washed with 5 ml phosphate buffered saline.

Inoculation Protocol

Tumor cells (4×10⁵ cells, in 30 μl, per animal) are injected into the pancreas using needle 30 G. Tumor cells inoculation to animals was performed under anesthesia (Ketamine 85 mg/kg & Xylasine 5 mg/kg).

Administration of Compositions:

Compositions were administered IP using needle 27 G. Compositions were administered daily from Day 4 for duration of 12 days (see Table I). Body weights measurements (twice weekly) were used for dosing volume calculations from recent measurement.

On study Day 18 animals were sacrificed by Carbon dioxide asphyxiation. Following termination of the study pancreas tumor from all animals was excised and weighed.

The administration of the test items to the different groups and end result of treatment is summarized in Table 3.

Results

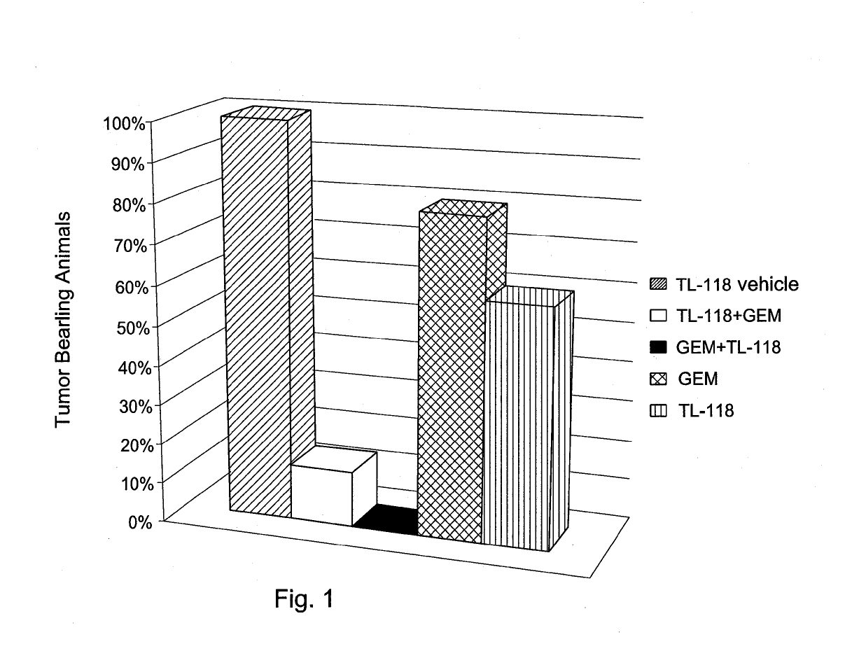

FIG. 1 shows the percentage of tumor bearing animals of each group treated according to schedule in Example 1. All subjects in treatment Group 1 developed tumors at the end of the study (100% of animals bore tumors). A moderate reduction in the number of tumor bearing animals was observed for Group 4 (about 80% of animals bore tumors) and Group 5 (about 50% of animals bore tumors). On the other hand Group 2 and Group 3 show dramatic reduction in the number of animal bearing tumors at the end of the scheduled treatment (about 10% for Group 2 and 0% for Group 3).

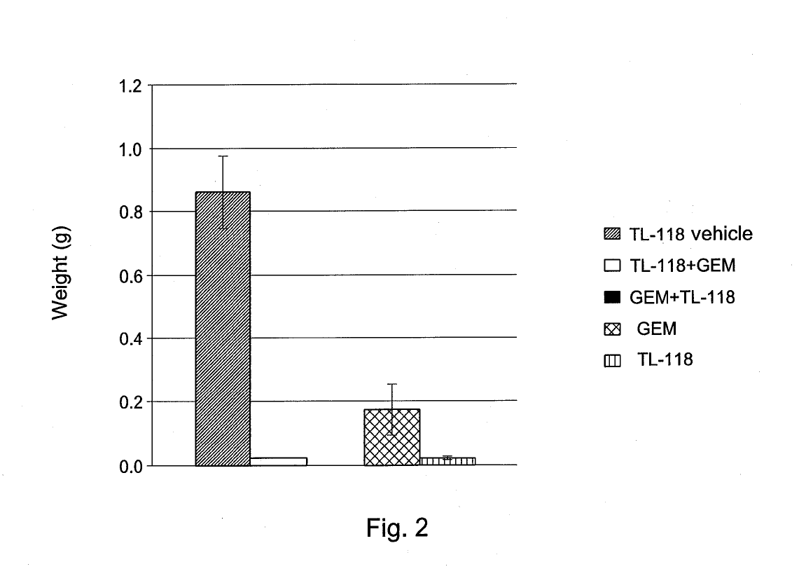

Similar results were shown when measuring average tumor weight of tumor-bearing animals of each group treated according to schedule in Example 1 (FIG. 2). Very significant reduction in average tumor weight was observed for Groups 2 and 3.

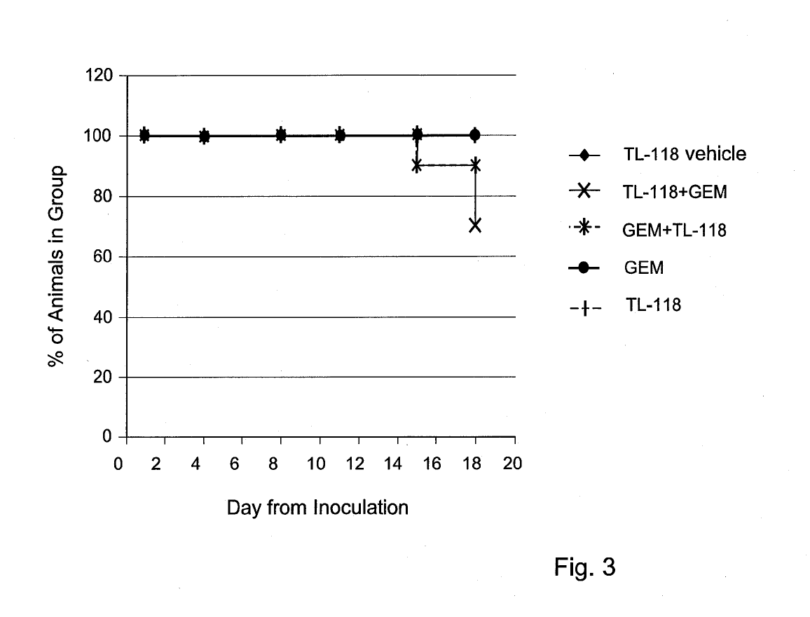

FIG. 3 shows animal survival of each group at each day during treatment schedule according to Example 1. Accordingly it is noted that significant reduction in animal survival was observed for Group 2, thus this treatment was terminated prior to day 18 of the schedule.

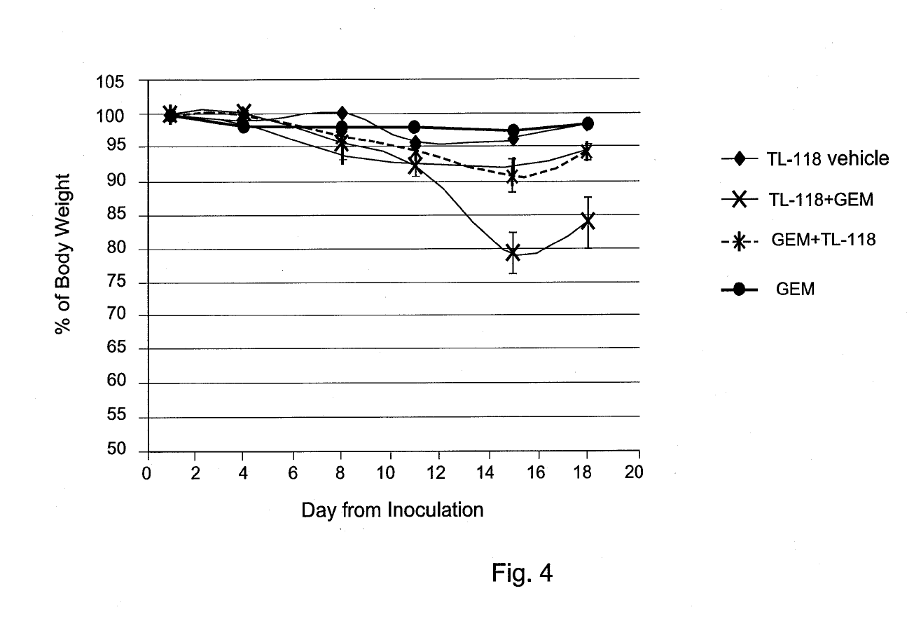

FIG. 4 shows animal body weight change at each day during treatment schedule according to Example 1. It is noted that apart from Group 2, all treatment group maintained % body weight of between 90 to 100% during the study.

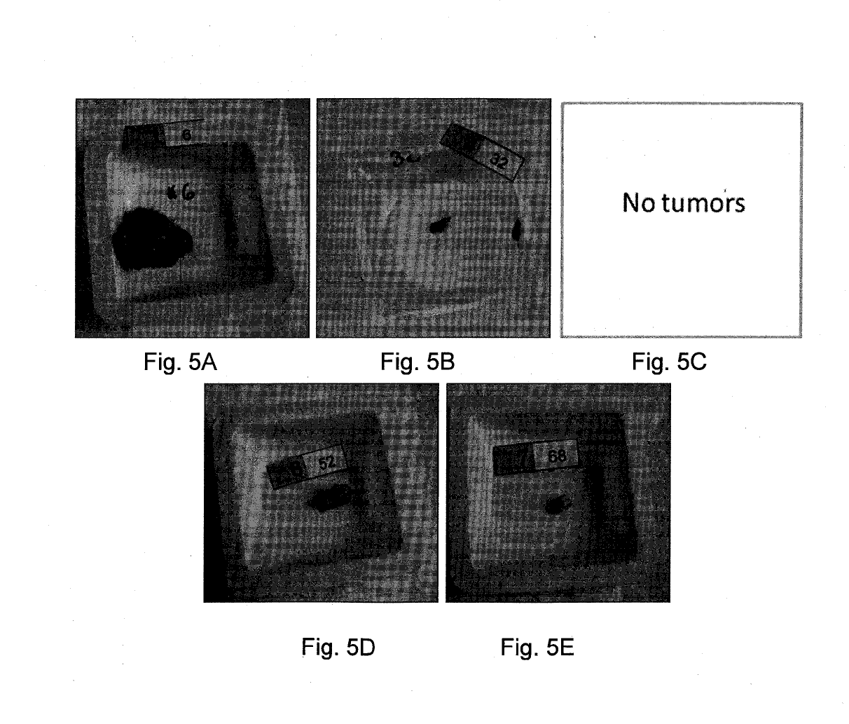

FIGS. 5A-5E illustrates representative tumors found at the end of each treatment schedule for each treatment group. As shown no tumors were found for treatment Group 5. Comparatively, large tumors were found in animals of Group 1, smaller volume tumors were found in animals of Groups 4 and 5.

TABLE 3
Treatment schedule and results

| | | Weekly Treatment Regime | | | | | | | |
|---|---|---|---|---|---|---|---|---|---|
| Group | Composition | Wed. | Thur. | Fri. | Sat. | Sun. | Mon. | Tue. | End Result Effect |
| 1 | Vehicle | + | + | + | − | + | + | + | very large tumors |
| 2 | Composition (a) | + | + | + | − | + | + | + | 6 out of 7 animals are |
| | Composition (b) | − | + | − | − | − | + | − | tumor free |
| 3 | Composition (a) | − | + | + | − | + | + | + | 9 out of 9 animals are |
| | Composition (b) | + | − | − | − | + | − | − | tumor free |
| 4 | Composition (b) | + | − | − | − | + | − | − | small tumors relative |
| | | | | | | | | | to vehicle, 2 out of |
| | | | | | | | | | 10 animals tumor free. |
| 5 | Composition (a) | + | + | + | − | + | + | + | very small tumors |
| | | | | | | | | | relative to vehicle, 4 |
| | | | | | | | | | out of 10 animals tumor |
| | | | | | | | | | free. |

1-44. (canceled)

45. A combination comprising:
a. a composition comprising at least one non-steroidal anti-inflammatory agent and at least one cytotoxic agent; and
b. a composition comprising at least one ribonucleotide reductase inhibitor.

46. The combination according to claim 45, wherein composition (a) further comprises at least one of: levamisol, an NFkB inhibitor, an H2-blocker, at least one agent that enhances intracellular accumulation of NADH+H⁺, an inhibitor of a matrix metalloproteinase, an inhibitor of a pro-angiogenic factor, a retinoid and a redox quinone.

47. The combination according to claim 45, wherein said at least one cytotoxic agent is selected from the group consisting of: cyclophosphamide, ifosfamide, cytarabine, 6-mercaptopurine, 6-thioguanine, vincristine, doxorubicin, daunorubicin, chlorambucil, carmustine, vinblastine, methotrexate, mitoxantrone, paclitaxel or their pharmaceutically acceptable salts, and any and any metabolite, prodrug or combination thereof.

48. The combination according to claim 45, wherein said at least one non-steroidal anti-inflammatory agent is selected from a group consisting of COX-1 and COX-2 inhibitors.

48. The combination according to claim 45, wherein said at least one non-steroidal anti-inflammatory agent is selected from the group consisting of diclofenac, piroxicam, indomethacin and any combination thereof.

50. The combination according to claim 46, wherein said agent enhancing intracellular accumulation of NADH+H⁺ is a poly-alcohol.

51. The combination according to claim 46, wherein said H2-blocker is selected from the group consisting of cimetidine, ranitidine, famotidine and nizatidine.

52. The combination according to claim 46, wherein said NFkB inhibitor is sulfasalazine or rapamycin.

53. The combination according to claim 45, wherein said at least one ribonucleotide reductase inhibitor is selected from fludarabine, cladribine, gemcitabine, tezacitabine, triapine, motexafin gadolinium, hydroxyurea, gallium maltolate, gallium nitrate and any metabolite, prodrug or combination thereof.

54. A method of treating cancer in a mammal comprising administering to said mammal:
a. a composition comprising at least one non-steroidal anti-inflammatory agent and at least one cytotoxic agent; and
b a composition comprising at least one ribonucleotide reductase inhibitor.

55. The method according to claim 54, wherein composition (a) and composition (b) are administered sequentially or concomitantly.

56. The method according to claim 54, wherein said compositions are administered during a one week cycle.

57. The method according to claim 54, wherein composition (a) is administered prior to the administration of composition (b).

58. The method according to claim 54, wherein composition (b) is administered prior to the administration of composition (a).

59. The method according to claim 54, wherein composition (a) is administered twice a week and composition (b) is administered twice a week.

60. The method according to claim 54, wherein composition (a) is administered twice a week and composition (b) is administered once a week.

61. The method according to claim 54, further comprising administration of composition (c) comprising an H2-blocker and an NFkB inhibitor.

62. A kit comprising: a first container comprising composition (a) comprising at least one non-steroidal anti-inflammatory agent and at least one cytotoxic agent; a second container comprising composition (b) comprising at least one ribonucleotide reductase inhibitor; and instructions for administration.

63. A kit according to claim 62, further comprising a third container comprising composition (c) comprising H2-blocker and an NFkB inhibitor.

64. A method according to claim 61, wherein said compositions are administered in the following weekly cycle:

| Day | Composition administered |
|---|---|
| 1 | Composition (b) |
| 2 | Composition (a) |
| 3 | Composition © |
| 4 | Compositi © (c) |
| 5 | Composition (a) |
| 6 | Compos ©on (c) |
| 7 | Composition (c) |
